(19) United States
(12) Patent Application Publication
(10) Pub. No.: US 2019/0167903 A1
(43) Pub. Date: Jun. 6, 2019

(54) DEVICE FOR ADMINISTERING A DOSE OF A FLUID PRODUCT

(71) Applicant: TECPHARMA LICENSING AG, Burgdorf (CH)

(72) Inventors: Seline Staub, Volketswil (CH); Jürg Hirschel, Bern (CH)

(21) Appl. No.: 16/257,664

(22) Filed: Jan. 25, 2019

Related U.S. Application Data
(63) Continuation of application No. PCT/CH2017/000063, filed on Jun. 21, 2017.

(30) Foreign Application Priority Data
Jul. 26, 2016 (CH) 00960/16

(51) Int. Cl. A61M 5/20; A61M 5/315

(52) U.S. Cl. CPC A61M 5/2033; A61M 5/2448; A61M 5/3158

(57) ABSTRACT

A device for administering a dose of a fluid product, where, in a starting state of the device, a holding element of the device is spaced apart in the proximal direction from a proximal end or portion of the housing and, moreover, a discharge stop of a drive member of the device is provided, where the discharge stop of the drive member can come into abutting contact with a discharge stop of the housing, and the discharge stop of the drive member is arranged on the drive member in a position axially offset in the proximal direction with respect to an engagement element of the drive member.

### CROSS-REFERENCE TO RELATED APPLICATIONS

This application is a continuation of International Application No. PCT/CH2017/000063 filed Jun. 21, 2017, which claims priority to Swiss Application No. 00960/19 filed Jul. 26, 2016, the entire contents of all of which are incorporated by reference herein in their entirety.

### FIELD OF THE INVENTION

The invention relates to a device for administering a dose of a fluid product.

### BACKGROUND

The term “medicine” here includes any flowable medicinal formulation that is suitable for controlled administration through a means, e.g., a cannula or hollow needle, for example, comprising a fluid, a solution, a gel, or a fine suspension that contains one or more active medicinal ingredients. A medicine can be a compound with a single active ingredient or a pre-mixed or co-formulated compound with multiple active ingredients from a single container. Medicines include drugs such as peptides (e.g., insulins, medicines containing insulin, preparations, derived preparations or analog preparations containing GLP-1), proteins and hormones, biologically obtained or active ingredients, active ingredients based on hormones or genes, nutritional formulations, enzymes, and other substances both in solid (suspended) or fluid form, but also polysaccharides, vaccines, DNA or RNA or oligonucleotides, antibodies or parts of antibodies and suitable base substances, additives, and carrier substances.

### SUMMARY OF THE INVENTION

Such devices are known from the prior art. For example, from WO 2009/100550 A1, a device for administering a dose is known.

Implementations provide an alternative device for administering a dose, wherein the dosing accuracy is increased.

For instance, a device for administering a dose of a fluid product is provided in independent claim 1. Preferred embodiments of such a device are given in the dependent claims.

In the following, the proximal direction for a device means the direction toward the housing-side end, and the distal direction for a device means the direction toward the needle-side end.

The invention relates to a device for administering a dose of a fluid product, with a housing that has a distal and a proximal end or section. The device also has a pretensioned spring that is held at least partially by a drive element. The drive element is supported so that it can move axially in the housing, in order to move a plug held in a cartridge, in particular, in a multi-chamber cartridge, in the distal direction for administering the dose of the fluid product. For limiting the discharge or distribution of a dose from the device, a discharge stop is provided on the housing, which can be brought into stop contact with a discharge stop provided on the drive element. In an especially preferred way, the discharge stop of the drive element can be brought into axial stop contact with the discharge stop of the housing. The dose to be dispensed or discharged can preferably be a fixed dose. The device further comprises a retaining element for retaining the drive element, loaded with a force of the spring. The retaining element and/or the housing can have a sleeve-shaped construction. The retaining element can be held at least partially by the housing. The spring can be supported on the distal end on the drive element and on the proximal end on the retaining element. In a starting state of the device, in which the device is delivered, the drive element can be held in a proximal position by an engagement between an engagement element provided on the retaining element and an engagement element provided on the drive element, wherein, in a released state of the device, wherein the discharge or distribution of the dose from the device is released, the engagement of the retaining element is out of engagement with the engagement element of the drive element. At least in the starting state of the device, the retaining element is spaced apart in the proximal direction from the proximal end or section of the housing, in particular, from a proximal housing stop that is provided on the proximal end or section of the housing. The proximal housing stop can be provided preferably on an inner lateral surface of the housing. The retaining element can be deformed and/or expanded at least in the starting state in the proximal direction relative to the housing. In the released state of the device, the retaining element can be brought into stop contact in the proximal direction with the proximal end or section of the housing, especially on the proximal housing stop, due to the force of the spring acting in the proximal direction. The discharge stop of the drive element is arranged on the drive element offset axially in the proximal direction relative to the engagement element of the drive element.

By means of the arrangement and/or construction of the device according to the invention, a spring with a large force can be held in the device or a fluid product with a high viscosity can be administered or a large dose quantity of a fluid product can be discharged, without the dosing accuracy being negatively affected.

In an especially preferred way, the discharge stop of the drive element can also be arranged on the drive element offset in the circumferential direction by 90 degrees relative to the engagement element of the drive element. The drive element can preferably have a sleeve-shaped construction. The drive element is constructed such that it can at least partially hold the pretensioned spring. The retaining element is constructed such that the retaining element can partially hold the drive element at least in the starting state.

The proximal end of the drive element is preferably in the starting state of the device in stop contact with the housing, in particular, with the proximal end or section of the housing. In an especially preferred way, the proximal end of the drive element in the starting state of the device is in stop contact with the proximal housing stop of the housing. The retaining element can also have a connecting piece that is arranged, at least in the starting state of the device, in a notch provided on the proximal end of the drive element. This arrangement can be used such that the retaining element and the drive element are supported locked in rotation relative to each other. This arrangement can also be used such that the retaining element in the starting state of the device is spaced apart from the proximal end or section of the housing, in particular, from the proximal housing stop of the housing. However, other arrangements and constructions of the device are also possible, wherein the retaining element in the starting state of the device is spaced apart from the proximal end or section of the housing, in particular, from the proximal housing stop of the housing.

The engagement element of the retaining element can preferably be constructed as a cam. The retaining element can have multiple engagement elements. In an especially preferred way, the retaining element comprises two engagement elements or cams, each of which is arranged on an arm of the retaining element that can be tensioned, in particular, on an arm of the retaining element that can be deflected elastically radially outward or on an arm of the retaining element that can be deflected elastically radially inward. The arms, in particular, the two arms of the retaining element, can preferably be connected to each other by means of the connecting piece of the retaining element.

The engagement element of the drive element can preferably be constructed as a recess. The drive element can have multiple engagement elements. In an especially preferred way, the drive element comprises two engagement elements or recesses.

Alternatively, the engagement element of the drive element can be constructed as a cam and the engagement element of the retaining element can be constructed as a recess.

The engagement element of the retaining element or the drive element can also have a different construction, if the engagement element of the retaining element and the engagement element of the drive element can be detachable in engagement. Preferably, the engagement element of the retaining element and/or the engagement element of the drive element are constructed such that a deformation and/or expansion of the engagement element of the retaining element and/or the engagement element of the drive element is reduced or prevented, if the engagement element of the retaining element and/or the engagement element of the drive element are in engagement and a force of the spring acts on the drive element and on the retaining element. In an especially preferred way, the drive element and/or the retaining element are constructed such that a deformation and/or expansion of the drive element and/or retaining element is reduced or prevented if a force of the spring acts on the drive element and on the retaining element.

Preferably, the outer diameter of the drive element on the distal end of the drive element and on the proximal end of the drive element are also different, wherein the inner diameter of the drive element on the distal end and on the proximal end of the drive element is the same. The size of the inner diameter of the drive element is selected such that the spring can be at least partially held by the drive element. In an especially preferred way, the outer diameter of the drive element is larger on the proximal end than on the distal end of the drive element, wherein the outer diameter of the distal end of the drive element is constructed such that the distal end of the drive element extends into the cartridge, in particular, into the multi-chamber cartridge, when the discharge stop of the drive element is in stop contact with the discharge stop of the housing. By means of this corresponding adaptation of the wall thickness of the drive element, the dose-relevant expansion or dose-relevant compression of the drive element can be reduced or even eliminated.

The retaining element can have an element for guiding the spring. The spring is preferably constructed as a coil spring. The coil-shaped spring is arranged around the element of the retaining element.

In order to activate the device, the device preferably has an actuation element that can be moved relative to the housing. The actuation element is preferably arranged so that it can move axially relative to the housing. The actuation element can comprise a recess into which the engagement element of the retaining element can extend in the released state of the device. Here, the engagement element of the retaining element is led out of engagement with the engagement element of the drive element. The drive element is consequently released by the retaining element and can move relative to the housing in the distal direction due to the force of the spring.

The device also comprises a holding device for holding the fluid product, which is supported so that it can be rotated or screwed relative to the housing. The holding device can have a cartridge, in particular, a multi-chamber cartridge. In an especially preferred way, the holding device can have a multi-chamber cartridge, wherein the multi-chamber cartridge can be constructed preferably as a two-chamber cartridge with two plugs and a bypass. So that the multi-chamber cartridge, in particular, the two-chamber cartridge, can be held in a defined position relative to the holding device, there is preferably a retaining arm, in particular, two retaining arms, on a distal end of the drive element. The retaining arms of the drive element hold the multi-chamber cartridge, in particular, the two-chamber cartridge, without play in the holding device. The two retaining arms can preferably form a tappet for driving the two plugs. Alternative constructions of the drive element are also possible. In particular, the drive element may have no retaining arms.

The holding device can be inserted or screwed into the housing by means of a threaded connection between the holding device and the housing. By inserting or screwing in the holding device, a plug can be moved within the two-chamber cartridge in the distal direction relative to the two-chamber cartridge, wherein initially the drive force can be transferred to the other plug by means of a solvent provided between the two plugs, so that both plugs can be driven in the distal direction. As soon as the first plug enters the area of the bypass of the two-chamber cartridge, the first plug can remain in place relative to the two-chamber cartridge. In contrast, the second plug can be driven farther, so that the solvent can be moved through the bypass, in order to release the lyophilized active ingredient held in the two-chamber cartridge. The device is in a mixed state when the solvent has completely activated the active ingredient.

By inserting or screwing the holding device farther into the housing, both plugs can be simultaneously moved in the distal direction. By means of a cannula of an injection needle unit that is provided on the device, unnecessary air can escape from the two-chamber cartridge. Optional axial play between the two-chamber cartridge and the holding device and the housing can be further reduced or even eliminated, so that safe, reliable, and more precise administration of the fluid product can be guaranteed. The device is thus in a deaerated state.

The device can also comprise a blocking element. The blocking element is preferably arranged so that it can rotate relative to the housing of the device. The blocking element can be moved from a blocking position, in which the actuation element is prevented from axial movement relative to the housing, into a release position, in which the actuation element can move axially relative to the housing. For this purpose, the holding device and the blocking element can each have a stop, wherein both stops interact for moving the blocking element from the blocking position into the release position. By inserting or screwing the holding device into the housing, the holding device can move the blocking element with it. Preferably, in this way, the blocking element can be rotated relative to the housing. Preferably, at the end of the mixed state of the device, by inserting or screwing the holding device farther into the housing, an extension provided on the actuation element can be moved into axial alignment relative to a recess provided on the blocking element. Alternatively, during the mixing of the solvent with the active ingredient, the blocking element can be moved into the release position. In the release position of the blocking element, the actuation element can be moved along the housing longitudinal axis relative to the housing. In the blocking position, an extension edge of the actuation element provided on the extension can be moved into stop contact with a blocking element edge provided on the blocking element, in order to prevent a relative movement of the actuation element along the housing longitudinal axis relative to the housing. The release position of the blocking element preferably corresponds to a position of the multi-chamber cartridge, in particular, the two-chamber cartridge, in which the multi-chamber cartridge, in particular, the two-chamber cartridge, is mixed or deaerated.

The blocking element ensures that the device can be actuated only shortly before the application. Especially with multi-chamber cartridges, in particular, with two-chamber cartridges, premature actuation of the device should be prevented. Above all, it should be ensured that the multi-chamber cartridge, in particular, the two-chamber cartridge, has been completely mixed or has been completely mixed and deaerated before a dose can be released.

Alternatively, the device can be provided with other elements that prevent unintentional or premature actuation of the device.

A corresponding indication device can also be provided in the form of a digit, a letter, or another symbol on the holding device, wherein, through a window provided on the housing, the starting state and/or the mixed state and/or the deaerated state of the device can be indicated.

The holding device can also have one or more latching arms that can latch in engagement with one or more latching openings or latching longitudinal grooves provided in the housing, in order to form a device that prevents backward movement or backward rotation, so that a safe and reliable administering of the fluid product can be guaranteed. The device that prevents backward movement or backward rotation can also generate an acoustic signal, in order to indicate which state the device is in.

An arm that can be latched in a recess of the holding device in a latched state of the device can also be provided on the drive element. This latching can also generate an acoustic sound, in order to indicate that the device is in the latched or used state. The device can also preferably no longer be used due to the engagement between the arm of the drive element and the recess of the holding device.

In order to be able to assemble a device according to the invention, a retaining element can be inserted with an engagement element into the housing in the proximal direction. A blocking element can be guided by means of one or more guides into the housing in the distal direction. A drive element that is held with a spring and has a retaining element can then be inserted into the housing in the proximal direction and rotated or screwed in relative to the housing, wherein an element provided on the retaining element is used for guiding the spring. Then an actuation element can be inserted into the housing in the distal direction. A holding device that has a cartridge, in particular, a multi-chamber cartridge, can be screwed into the housing in the proximal direction, such that the device is in a starting state.

### BRIEF DESCRIPTION OF THE DRAWINGS

The invention is described below with reference to several figures. The features disclosed here are advantageous refinements of the invention. Shown are:

FIG. 1, an exploded view diagram of a first embodiment of a device according to the invention,

FIG. 2a, an external view of the device from FIG. 1 in a starting state in which the device is delivered,

FIG. 2b, a longitudinal section view of the device according to FIG. 2a, wherein the longitudinal section view corresponds to the section line A-A shown in FIG. 2a,

FIG. 2c, a longitudinal section view of the device from FIG. 1, wherein the view is rotated by 90 degrees relative to that of FIG. 2a,

FIG. 2d, a detail view of the device from FIG. 1, in which the blocking element and the actuation element and their interaction in the starting state can be seen,

FIG. 3a, an external view of the device from FIG. 1 in a mixed state,

FIG. 3b, a longitudinal section view of the device according to FIG. 3a, wherein the longitudinal section view corresponds to the section line A-A shown in FIG. 3a,

FIG. 3c, a longitudinal section view of the device from FIG. 1, wherein the view is rotated by 90 degrees relative to that of FIG. 3a,

FIG. 3d, a detail view of the device from FIG. 1, in which the holding device, the blocking element, and the actuation element and their interaction in the mixed state can be seen,

FIG. 4a, an external view of the device from FIG. 1 in a deaerated state,

FIG. 4b, a longitudinal section view of the device according to FIG. 4a, wherein the longitudinal section view corresponds to the section line A-A shown in FIG. 4a,

FIG. 4c, a longitudinal section view of the device from FIG. 1, wherein the view is rotated by 90 degrees relative to that of FIG. 4a,

FIG. 4d, a detail view of the device from FIG. 1, in which the holding device, the blocking element, and the actuation element and their interaction in the deaerated state can be seen,

FIG. 5a, an external view of the device from FIG. 1 in a released state, wherein a discharge or distribution of a dose from the device is released,

FIG. 5b, a longitudinal section view of the device according to FIG. 5a, wherein the longitudinal section view corresponds to the section line A-A shown in FIG. 5a,

FIG. 5c, a longitudinal section view of the device from FIG. 1, wherein the view is rotated by 90 degrees relative to that of FIG. 5a,

FIG. 5d, a detail view of the device from FIG. 1, in which the blocking element and the actuation element and their interaction in the released state can be seen,

FIG. 6a, an external view of the device from FIG. 1 in a discharged state,

FIG. 6b, a longitudinal section view according to FIG. 5a, wherein the longitudinal section view corresponds to the section line A-A shown in FIG. 6a, and

FIG. 6c, a longitudinal section view from FIG. 1, wherein the view is rotated by 90 degrees relative to that of FIG. 5a.

### DETAILED DESCRIPTION

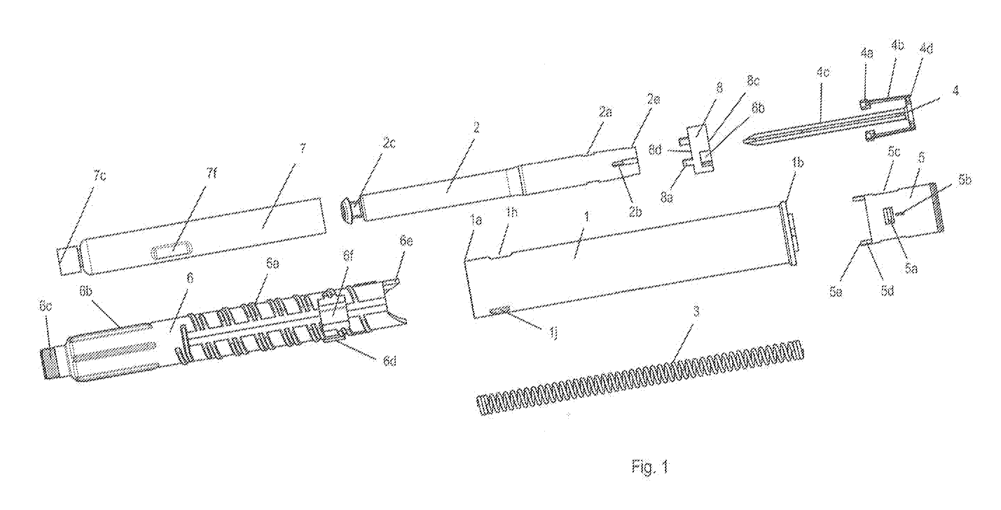

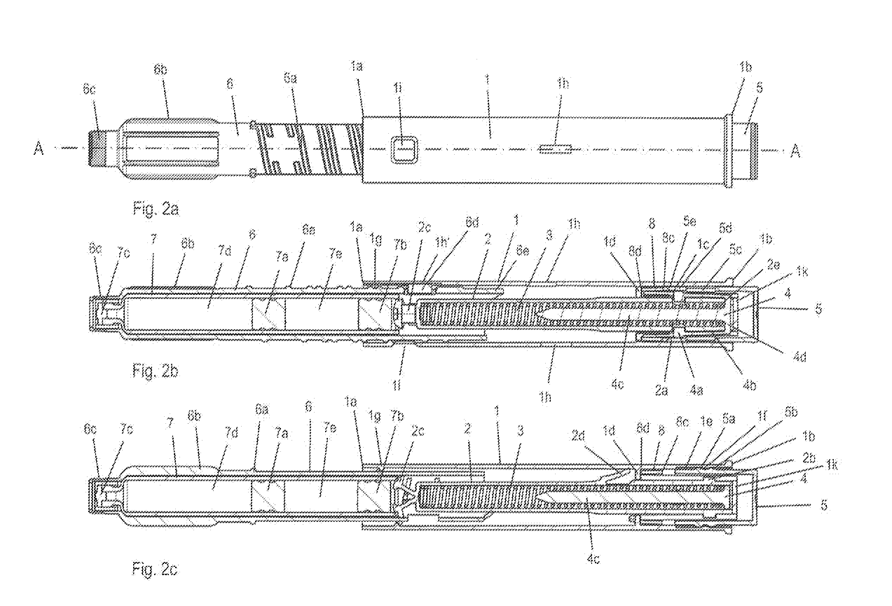

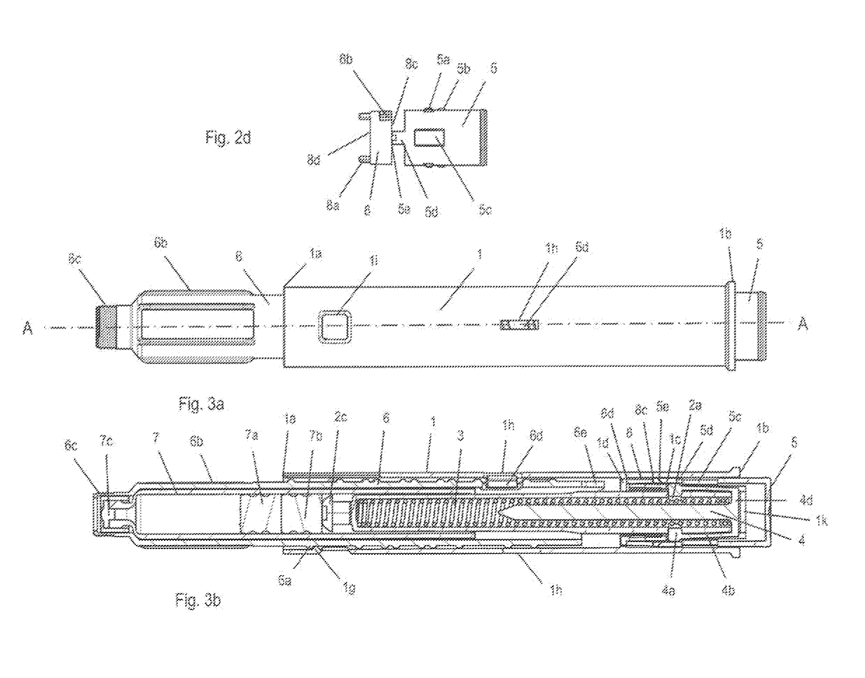

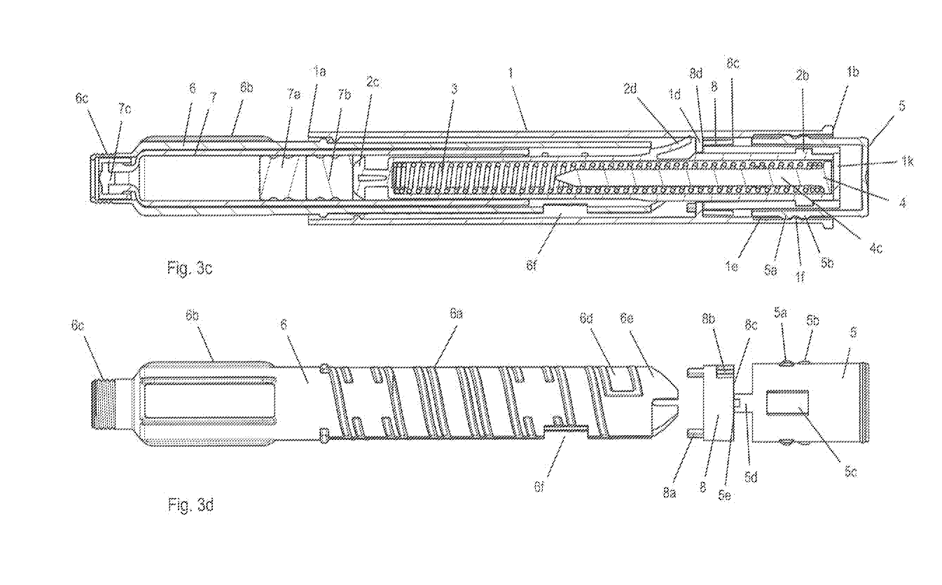

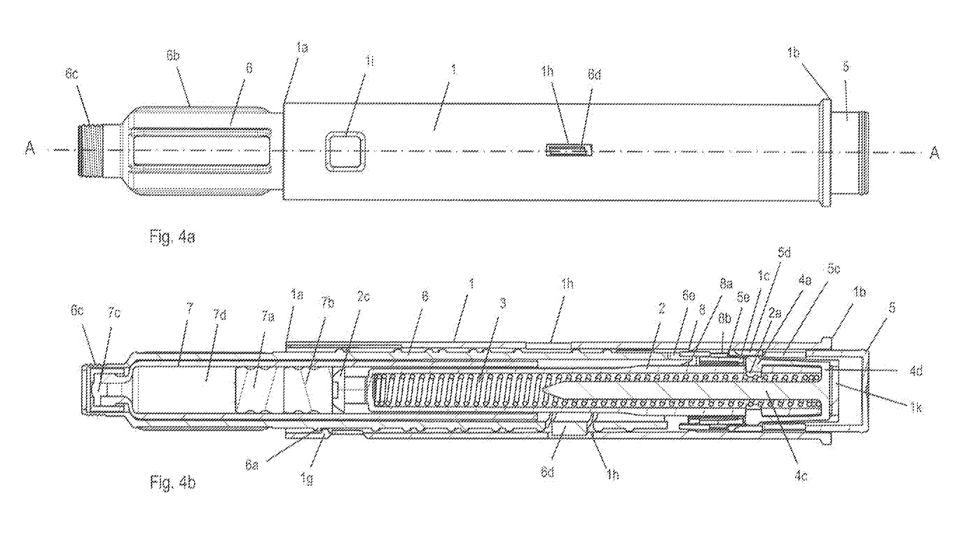

In FIG. 1, an exploded view diagram of a first embodiment of the device according to the invention is shown. The device comprises a housing (1) with a distal end (1a) and a proximal end (1b) or section. The housing (1) has a sleeve-shaped construction. The proximal end (1b) or section of the housing further comprises a proximal housing stop (1k; FIG. 2b). The proximal housing stop (1k; FIG. 2b) is provided on an inner lateral surface of the proximal end (1b) or section of the housing (1). One or more cams (1j) can be provided on an outer lateral surface of the housing (1), which are used to prevent the device from rolling, for example, on a table. The device also comprises a drive element (2), wherein the drive element (2) is supported so that it can move in the axial direction in the housing (1). The drive element (2) has a sleeve-shaped construction. The outer diameter of the drive element (2) can preferably be different on the distal end and on the proximal end, wherein the inner diameter of the drive element on the distal end and on the proximal end of the drive element is the same. The size of the inner diameter of the drive element (2) is constructed such that a pretensioned spring (3) is at least partially held by the drive element (2). In an especially preferred way, the outer diameter of the drive element (2) is larger on the proximal end than on the distal end. In addition, the device comprises a retaining element (4). The retaining element (4) is used for retaining the drive element (2) loaded with a force of the spring. The pretensioned spring (3) is supported on a distal end on the drive element (2) and on a proximal end on the retaining element (4). An engagement element (4a) that is preferably formed as a cam (4a) is provided on the retaining element (4). The retaining element (4) has an arm (4b) that can be tensioned, in particular, an arm (4b) that can be deflected elastically radially outward or an arm that can be deflected elastically radially inward, on which the engagement element (4a), in particular, the cam (4a) is arranged. In an especially preferred way, the retaining element (4) has two or more engagement elements (4a), in particular, two or more cams (4a) that are arranged on corresponding arms (4b) of the retaining element (4). In an especially preferred way, the retaining element (4) comprises one or more connecting pieces (4d), each of which connect two arms (4b) of the retaining element (4) to each other. On the drive element (2) there is an engagement element (2a) that is preferably formed as a recess (2a). In an especially preferred way, the drive element (2) has two or more engagement elements (2a), in particular, two or more recesses (2a). The engagement element (4a) of the retaining element (4) and the engagement element (2a) of the drive element (2) are constructed such that, in a starting state of the device, the drive element (2) can be held in a proximal position by an engagement between the engagement element (4a) of the retaining element (4) and the engagement element (2a) of the drive element (2). The engagement element (4a) of the retaining element (4) and the engagement element (2a) of the drive element (2) are also constructed such that, in a released state of the device, the engagement element (4a) of the retaining element (4) can be brought out of engagement with the engagement element (2a) of the drive element (2). The retaining element (4) is, at least in the starting state, spaced apart in the proximal direction from the proximal end (1b) or section of the housing (1), in particular, from the proximal housing stop (1k; FIG. 2b). The retaining element (4) and the drive element (2) can be supported so that they are locked in rotation relative to each other. For this purpose, the connecting piece (4d) of the retaining element (4) can be arranged, at least in the starting state of the device, in a notch (2e) provided on the proximal end of the drive element (2). In order to support the retaining element (4) and the housing (1) so that they are locked in rotation relative to each other, the arm (4b) of the retaining element (4) can be held in a longitudinal recess (not visible) or longitudinal groove (not visible) provided on the housing (1). On the housing (1) there is also a discharge stop (1d; FIG. 2c) that can be brought into stop contact with a discharge stop (2b) formed on the drive element (2), in order to administer a dose. Alternatively, there can also be multiple discharge stops (1d; FIG. 2c) of the housing (1) and multiple discharge stops (2b) of the drive element (2). The discharge stop (2b) of the drive element (2) is arranged on the drive element (2) offset axially in the proximal direction relative to the engagement element (2a) of the drive element (2). The discharge stop (2b) of the drive element (2) is also arranged on the drive element (2) offset in the circumferential direction by 90 degrees relative to the engagement element (2a) of the drive element (2). In addition, the discharge stop (2b) of the drive element can be held in a longitudinal recess (not visible) or longitudinal groove (not visible) provided on the housing (1), in order to support the drive element (2) and the housing (1) in a rotationally locked manner relative to each other. The retaining element (4) also comprises an element (4c) for guiding the spring (3). The element (4c) of the retaining element (4) can have a mandrel-shaped construction. The spring (3) can preferably be formed as a coil spring (3). The coil-shaped spring (3) surrounds the element (4c) of the retaining element (4) such that the spring (3) can be guided by the element (4c) of the retaining element (4). The device further comprises an actuation element (5) for activating the device. The actuation element (5) can move axially relative to the housing (1). The actuation element (5) is arranged rotationally locked relative to the housing (1). For this purpose, the actuation element (5) has, on an outer lateral surface, a first cam (5a) that extends into a longitudinal groove (1e; FIG. 2c) provided on the inner lateral surface of the housing (1). A second cam (5b) that is arranged on the actuation element (5) offset in the proximal direction relative to the first cam (5a) is also provided on the actuation element (5), with this cam also extending into the longitudinal groove (1e; FIG. 2c) of the housing (1). The longitudinal groove (1e) of the housing (1) is divided by a rib (1f) arranged on the inner lateral surface of the housing into a distal and a proximal section. The first cam (5a) and the second cam (5b) of the actuation element (5) and the rib (1f) of the housing (1) are constructed and act such that a relative movement of the actuation element (5) relative to the housing (1) can be prevented in the proximal direction, in order to prevent faulty handling. In addition, the first cam (5a) and the second cam (5b) of the actuation element (5) and the rib (1f) of the housing (1) are constructed and act such that, together, a relative movement of the actuation element (5) relative to the housing (1) is possible in the distal direction, in order to discharge a dose from the device. The actuation element (5) also has a recess (5c) that is constructed such that the engagement element (4a), especially the cam (4a) of the retaining element (4), can extend into the recess (5c) of the actuation element (5). On the housing (1), an extension (1c; FIG. 2b) can also be provided, which is used to limit a relative movement of the retaining element (4) relative to the housing (1) in the distal direction. The device further comprises a holding device (6) for holding the fluid product. The holding device (6) is supported so that it can rotate relative to the housing (1). On an outer lateral surface of the holding device (6) there is an outer thread (6a) that is in thread engagement with an inner thread (1g; FIG. 2a) formed on the inner lateral surface of the housing (1). On the outer lateral surface of the holding device (6) there can preferably be one or more longitudinal ribs (6b) so that the user can insert or screw the holding device into the housing (1) with the help of the longitudinal ribs (6b) of the holding device (6). The holding device (6) can preferably have one or more latching arms (6d) that can latch in engagement with one or more latching openings (1h) provided in the housing, in order to ensure that the holding device (6) can be inserted or screwed into the housing (1) only in the distal direction. In this way, a device for preventing backward movement or backward rotation can be constructed, so that a safe and reliable administration of the fluid product can be guaranteed. An indication device (not visible) in the form of digits, letters, or other symbols can also be arranged on the holding device (6), with these symbols being visible through one or more windows (1i) provided on the housing. In this way it can be indicated which state the device is in. A multi-chamber cartridge (7), in particular, a two-chamber cartridge (7), is held in the holding device (6). The device preventing backward movement or backward rotation can also generate an acoustic signal in order to indicate which state the device is in. The drive element (2) also comprises an arm (2d) that can be tensioned, in particular, an arm (2d) that can be deflected elastically radially outward or an arm that can be deflected elastically radially inward, which can engage in a recess (6f) provided on the holding device (6). The arm (2b) of the drive element (2) can also generate an acoustic signal when engaged in the recess (6f) of the holding device (6), by means of which it can be indicated that the discharge of the fluid product has ended. At the distal end of the drive element (2) there are one or more, in an especially preferred way, two retaining arms (2c), which press, at least in the starting state of the device, the two-chamber cartridge (7) in the distal direction. The two-chamber cartridge (7) is held by means of retaining arms (2c) of the drive element (2) in a defined position relative to the holding device (6). An axial movement of the two-chamber cartridge (7) in the holding device (6) is prevented in this way. The two-chamber cartridge (7) has a first plug (7a) and a second plug (7b). The second plug (7b) closes the two-chamber cartridge (7) on the proximal end. On the distal end of the two-chamber cartridge (7) there is a membrane (7c). The membrane (7c) can be pierced by a cannula (not visible) of an injection needle unit (not visible). For connecting the injection needle unit (not visible) with the device, on the distal end of the holding device (6) there is an injection needle unit connecting element (6c) in the form of an outer thread (6c) that can be in thread engagement with an inner thread (not visible) arranged on the injection needle unit (not visible). Alternatively, other connections between the holding device (6) and the injection needle unit (not visible) can be provided, for example, a snap-together connection or a latching connection. Between the membrane (7c) and the first plug (7a) of the two-chamber cartridge (7) there is a first chamber (7d), in which a lyophilized active ingredient is held. Between the first plug (7a) and the second plug (7b), a second chamber (7e) of the two-chamber cartridge (7) is formed, in which the solvent for the active ingredient is stored. The two-chamber cartridge (7) further comprises a bypass (7f), wherein the solvent can be brought via the bypass (7f) into the first chamber (7d), in order to mix the active ingredient with the solvent. The device further comprises a blocking element (8). The blocking element (8) has a sleeve-shaped construction. The blocking element (8) is arranged so that it can rotate and is axially fixed relative to the housing (1). A blocking element support (8d) provided on the blocking element (8) is supported on the housing (1). In order to position the blocking element (8) radially relative to the housing (1), a groove/cam connection (not visible) can be provided preferably between the outer lateral surface of the blocking element (8) and the inner lateral surface of the housing (1). The blocking element (8) is provided for blocking the actuation element (5), wherein by rotating the holding device (6) relative to the housing (1), the blocking element (8) can move from a blocking position into a release position. The holding device (6) has one or more stops (6e) that can be brought into stop contact with one or more stops (8a) provided on the blocking element (6). The stop (8a) of the blocking element (8) extends from the blocking element support (8d) of the blocking element (8) in the distal direction. The stop (6e) of the holding device (6) extends from the proximal end of the holding device (6) in the proximal direction. The stop (6e) of the holding device (6) can interact with the stop (8a) of the blocking element (8) such that the blocking element (8) can be brought from the blocking position into the release position. The actuation element (5) and the blocking element (8) are constructed such that the actuation element (5) can be moved along a housing longitudinal axis relative to the housing (1) or can be fixed axially relative to the housing (1). The actuation element (5) can have one or more extensions (5d) that can be held in one or more recesses (8b) of the blocking element (8), wherein the actuation element (5) can move along the housing longitudinal axis of the housing (1). The extension (5d) of the actuation element (5) is arranged on a distal end of the actuation element (5) and extends in the distal direction. The recess (8b) is arranged on a proximal end of the blocking element (8) and extends in the distal direction. Furthermore, on the actuation element (5) there can be an extension edge (5e), in particular, on the extension (5d) of the actuation element (5), an extension edge (5e) that can be brought into stop contact with a blocking element edge (8c) arranged on the proximal end of the blocking element (8), in order to prevent movement of the actuation element (5) along the housing longitudinal axis relative to the housing (1).

In FIG. 2b, a longitudinal section view of the device from FIG. 1 is shown in a starting state, in which the device is delivered, wherein the longitudinal section view corresponds to the section line A-A drawn in FIG. 2a. A longitudinal section view of the device according to FIG. 2b is also shown in FIG. 2c, wherein the longitudinal section view is rotated by 90 degrees relative to that of FIG. 2a. The engagement element (4a), in particular, the cam (4a) of the retaining element (4), is in engagement with the engagement element (2a), in particular, with the recess (2a) of the drive element (2). The actuation element (5) is arranged relative to the retaining element (4) such that the engagement element (4a), in particular, the cam (4a) of the retaining element (4), can be held in engagement with the engagement element (2a), in particular, with the recess (2a) of the drive element. The spring (3) is supported so that it is pretensioned on the distal end on the drive element (2) and on the proximal end on the retaining element (4). The actuation element (5) is also arranged relative to the housing (1) such that the rib (1f) of the housing (1) is arranged between the first cam (5a) and the second cam (5b) of the actuation element (5). The arm (4b) of the retaining element (4) is held pretensioned elastically radially inward by means of an inner lateral surface of the actuation element (5). The drive element (2) is loaded with a force of the spring (3) acting in the distal direction. The retaining element (4) is spaced apart from the proximal housing stop (1k) in the proximal direction. The engagement element (4a), in particular, the cam (4a) of the retaining element (4), is supported in the proximal direction on the drive element (2), in particular, on the proximal end of the engagement element (2a), in particular, on the recess (2a) of the drive element (2). The proximal end of the drive element (2) is in stop contact with the housing (1). The distal end of the drive element (2) is in stop contact with the two-chamber cartridge (7) held in the holding device (6). The connecting piece (4d) of the retaining element (4) is arranged in the notch (2e) of the drive element (2). The arm (2d) of the retaining element (2) is pretensioned radially inward by the inner lateral surface of the housing (1). The two retaining arms (2c) of the drive element (2) press the two-chamber cartridge (7) in the distal direction. The two-chamber cartridge (7) is held by means of retaining arms (2c) of the drive element (2) in a defined position relative to the holding device (6). Axial movement of the two-chamber cartridge (7) in the holding device (6) is therefore prevented. The two retaining arms (2c) of the drive element (2) extend laterally away from the longitudinal axis of the drive element (2). The two retaining arms (2c) of the drive element (2) are spread apart relative to each other and hold the two-chamber cartridge (7) in the holding device (6) without play. For this purpose, the inclined surfaces provided on the retaining arms (2c) of the drive element (2) are in stop contact with a proximal edge of the two-chamber cartridge (7). The holding device (6) is in engagement with the housing (1) by means of the thread connection between the outer thread (6a) of the holding device (6) and the inner thread (1g) of the housing (1). The indication device (not visible), in particular, an appropriate digit, character, or letter, is visible through the window (1i) provided on the housing (1) such that it can be indicated that the device is in its starting state. The latching arm (6d) of the holding device (6) is further latched in a latching opening (1h) of the housing or alternatively in a latching longitudinal groove (1h′) of the housing (1), in particular, in a latching opening (1h) or latching longitudinal groove (1h′) arranged on the distal end of the device, which is arranged on the inner lateral surface of the housing (1). The blocking element support (8d) of the blocking element (8) is supported on the housing (1). Furthermore, the blocking element (8) is positioned radially relative to the housing (1) by means of a groove/cam connection (not visible). The blocking element (8) is in the blocking position, in which an actuation of the actuation element (5) is blocked. The actuation element (5) cannot be moved along the housing axis relative to the housing. As is visible in FIG. 2d, the extension edge (5e) of the extension (5d) of the actuation element (5) is led into stop contact with the blocking element edge (8c) of the blocking element (8).

In FIG. 3b, a longitudinal section view of the device from FIG. 1 is shown in a mixed state, wherein the longitudinal section view corresponds to the section line A-A drawn in FIG. 3a. Furthermore, in FIG. 3c, a longitudinal section view of the device according to FIG. 3b is shown, wherein the longitudinal section view is rotated by 90 degrees relative to FIG. 3a. An injection needle unit (not visible) can be placed on the injection needle unit connecting element (6c) of the holding device (6). A cannula (not visible) of the injection needle unit (not visible) can pierce the membrane (7c) of the two-chamber cartridge (7). The holding device (6) held with the two-chamber cartridge (7) can be inserted or screwed into the interior of the housing (1) in the proximal direction relative to the housing (1) from the starting state according to FIG. 2b and FIG. 2c. For this purpose, the holding device (6) is inserted or screwed into the housing (1) by means of the thread connection between the outer thread (6a) of the holding device (6) and the inner thread (1g) of the housing (1). By inserting or screwing in the holding device (6), the inclined surfaces of the retaining arms (2c) of the drive element (2) slide on the proximal edge of the two-chamber cartridge (7), wherein the retaining arms (2c) of the drive element are moved radially inward in the direction of the longitudinal axis of the drive element (2). The retaining arms (2c) of the drive element (2) move inward toward each other and form a tappet for the second plug (7b) of the two-chamber cartridge (7). The retaining element (4) is spaced apart in the proximal direction from the proximal housing stop (1k). The engagement element (4a), in particular, the cam (4a) of the retaining element (4), is supported in the proximal direction on the drive element (2), in particular, on the proximal end of the engagement element (2a), in particular, the recess (2a) of the drive element (2). The proximal end of the drive element (2) is in stop contact with the housing (1). The connecting piece (4d) of the retaining element (4) is arranged in the notch (2e) of the drive element (2). By inserting or screwing the holding device (6) into the housing (1), the second plug (7b) of the two-chamber cartridge (7) is moved farther in the distal direction within the two-chamber cartridge (7). In this way, first the drive force is transferred by the solvent in the second chamber (7e) to the first plug (7a), so that both plugs (7a, 7b) are driven in the distal direction. As soon as the first plug (7a) is in the region of the bypass (7f) of the two-chamber cartridge (7), the first plug (7a) remains in place relative to the two-chamber cartridge (7). The second plug (7b), in contrast, is driven farther, so that the solvent is brought from the second chamber (7e) via the bypass (7f) into the first chamber (7d) and can release the active ingredient located there. The second plug (7b) is driven until it meets the first plug (7a). In this way, the mixed state of the device is reached. During this procedure, the holding device (6) is inserted or screwed farther into the housing (1). The indication device (not visible), in particular, a corresponding digit, a corresponding letter, or another corresponding character of the holding device (6) is visible through the window (1i) provided on the housing (1), such that it is indicated that the device is in the mixed state. Furthermore, the latching arm (6d) of the holding device (6) is latched in a latching opening (1h) of the housing (1), in particular, in a latching opening (1h) of the housing (1) arranged between the distal end and proximal end of the device.

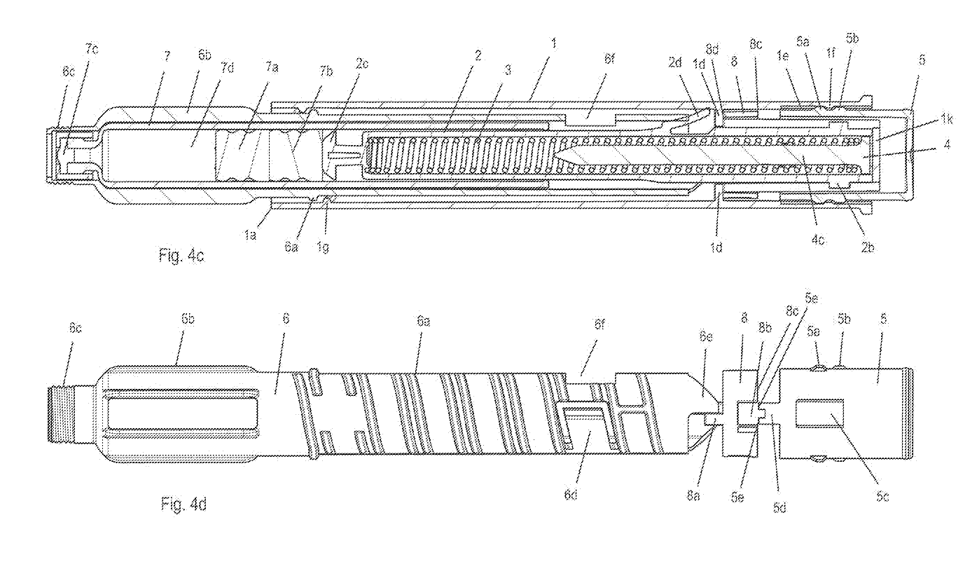

In FIG. 4b, a longitudinal section view of the device from FIG. 1 is shown in a deaerated state, wherein the longitudinal section view corresponds to the section line A-A drawn in FIG. 4a. A longitudinal section view of the device according to FIG. 4b is also shown in FIG. 4c, wherein the longitudinal section view is rotated by 90 degrees relative to that of FIG. 4a. From the mixed state according to FIGS. 3a and 3b, the holding device (6) is inserted or screwed farther into the housing (1) in the proximal direction. During further advance of the holding device (6) relative to the housing (1), both plugs (7a, 7b) are simultaneously pushed farther in the distal direction. Therefore, excess air can escape from the first chamber (7d) of the two-chamber cartridge (7) through the cannula (not visible) of the injection needle unit (not visible). In addition, optional axial play between the two-chamber cartridge (7) and the holding device (6) and the housing (1) can be reduced or even eliminated, so that a safe, reliable, and more precise administration of the fluid product can be guaranteed. The retaining element (4) is spaced apart from the proximal housing stop (1k) in the proximal direction. The engagement element (4a), in particular, the cam (4a) of the retaining element (4), is supported in the proximal direction on the drive element (2), in particular, on the proximal end of the engagement element (2a), in particular, the recess (2a) of the drive element (2). The proximal end of the drive element (2) is in stop contact with the housing (1). The connecting piece (4d) of the retaining element (4) is arranged in the notch (2e) of the drive element (2). As is visible in FIG. 4d, the blocking element (8) is moved from the blocking position into a release position. The stop (6e) of the holding device (6) is brought into stop contact with the stop (8e) of the blocking element (8). By inserting or screwing in the holding device (6), the holding device (6) takes along the blocking element (8), wherein the holding device (6) and the blocking element (8) are rotated relative to the actuation element (5). Here, the groove/cam connection (not visible) between the blocking element (8) and the housing (1) is released. At the end of the insertion movement or the screw-in movement of the holding device (6) into the housing (1), the extension (5d) of the actuation element (5) is in axial alignment with the recess (8b) of the blocking element (8). In the release position of the blocking element (8), the actuation element (5) is arranged relative to the blocking element (8) such that the actuation element (5) can be moved along the housing longitudinal axis relative to the housing (1). The indication device (not visible), in particular, a corresponding digit, a corresponding letter, or another corresponding character of the holding device (6) is visible through the window (1i) provided on the housing (1) such that it is indicated that the device is in the deaerated state. Furthermore, the latching arm (6d) of the holding device (6) is latched in a latching opening (1h) of the housing (1), in particular, in a latching opening (1h) of the housing (1) arranged on the proximal end of the device.

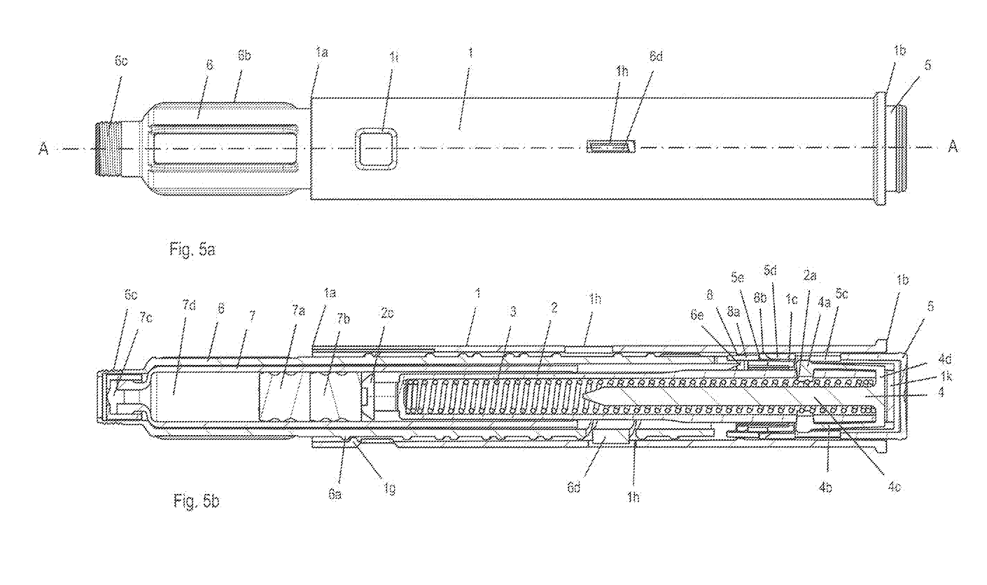

In FIG. 5b, a longitudinal section view of the device from FIG. 1 is shown in a released state, wherein the longitudinal section view corresponds to the section line A-A drawn in FIG. 5a. Furthermore, in FIG. 5c, a longitudinal section view of the device according to FIG. 5b is shown, wherein the longitudinal section view is rotated by 90 degrees relative to FIG. 5a. The actuation element (5) is pressed or moved in the distal direction relative to the housing (1). Here, the second cam (5b) of the actuation element (5) slides over the rib (1f) of the housing (1) and moves in the distal direction. As is visible in FIG. 5d, the extension (5d) of the actuation element (5) is brought into the recess (8b) of the blocking element (8). Furthermore, the recess (5c) of the actuation element (5) is moved via the engagement element (4a) of the retaining element (4), in particular, via the cam (4a) of the retaining element (4). Here, the engagement element (4a) of the retaining element (4), in particular, the cam (4a) of the retaining element (4), is moved out of engagement with the engagement element (2a) of the drive element (2), in particular, the recess (2a) of the drive element (2). Here, the elastically pretensioned arm (4b) of the retaining element (4) is released from tension, wherein the engagement element (4a) of the retaining element (4), in particular, the cam (4a) of the retaining element (4), is deflected radially outward. Here, the cam (4a) can slide along an inclined surface that is provided on the cam (4a), on an inclined surface provided on the recess (2a). The drive element (2) is released from the retaining element (4) and moves, due to the force of the spring (3), relative to the housing (1) in the distal direction. Due to the force of the spring (3) acting in the proximal direction, the retaining element (4) is moved in the proximal direction into stop contact with the proximal housing stop (1k). The connecting piece (4d) of the retaining element (4) is here moved out of engagement with the notch (2e) of the drive element (2).

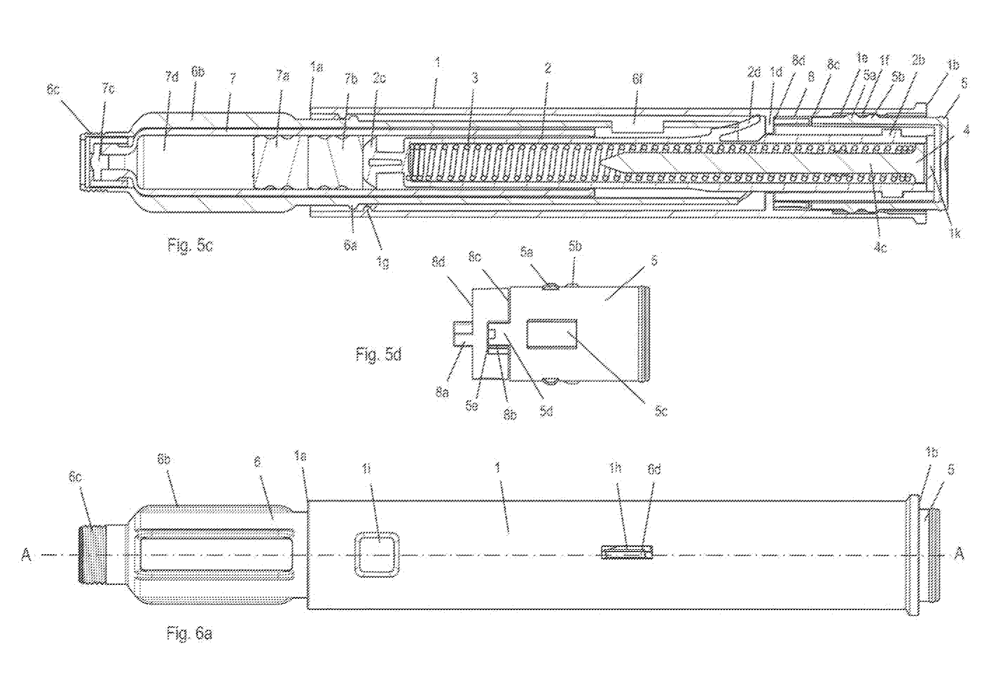

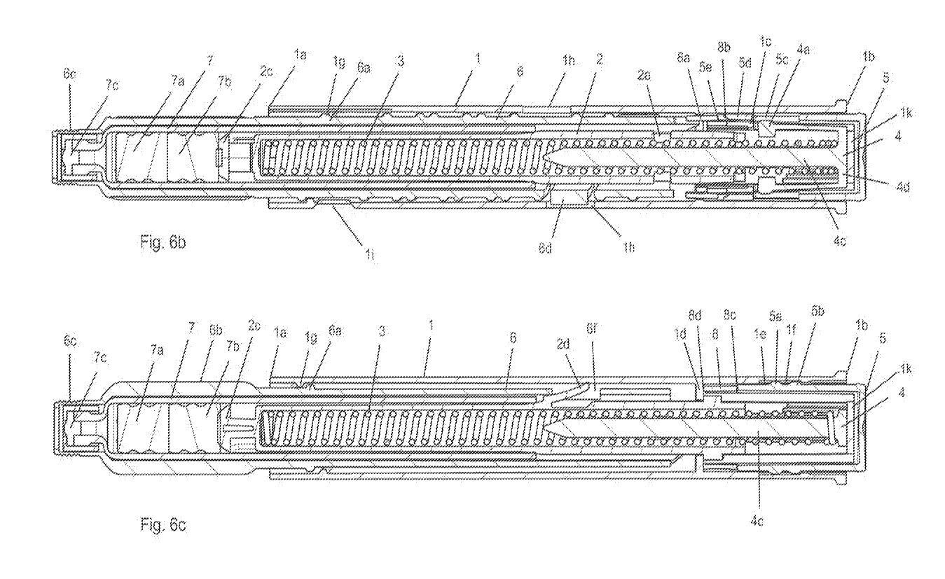

In FIG. 6b, a longitudinal section view of the device from FIG. 1 is shown in a latched state, wherein the longitudinal section view corresponds to the section line A-A drawn in FIG. 6a. Furthermore, a longitudinal section view of the device according to FIG. 6b is shown in FIG. 6c, wherein the longitudinal section view is rotated by 90 degrees relative to that of FIG. 6a. The drive element (2) is pushed by the force of the spring (3) relative to the two-chamber cartridge (7) and drives the first (7a) and the second plug (7b) within the two-chamber cartridge (7), so that the fluid product is discharged from the first chamber (7d) of the two-chamber cartridge (7). The spring (3) pushes the drive element (2) in the two-chamber cartridge (7) until the discharge stop (2b) of the drive element (2) is moved into stop contact with the discharge stop (1d) of the housing (1). The discharge stop (2b) of the drive element (2) is arranged between the engagement element (2a), in particular, the recess (2a) of the drive element (2) and the proximal end of the drive element (2). After the arm (2d) of the drive element (2) is further pretensioned radially inward by the walls of the cartridge holder (6), the radially inward pretensioned arm (2d) of the drive element (2) is moved into the recess (6f) of the holding device (6) at the end of the discharge of the fluid product. The arm (2) of the drive element can generate an acoustic noise in the latching procedure, in particular, in the tension relief process in the recess (6f) of the holding device (6), in order to indicate that the discharge has ended. The retaining element (4) is in stop contact with the proximal housing stop (1k) in the proximal direction due to the force of the spring (3) acting in the proximal direction.

### REFERENCE SYMBOLS

1 Housing
1a Distal end of the housing
1b Proximal end of the housing
1c Extension of the housing
1d Discharge stop of the housing
1e Longitudinal groove of the housing
1f Rib of the housing
1g Internal thread of the housing
1h Latching opening
1h′ Latching longitudinal groove
1i Window
1j Cam
1k Proximal housing stop
2 Drive element
2a Engagement element of the drive element, recess
2b Discharge stop of the drive element
2c Retaining arms
2d Arm
2e Notch
3 Spring
4 Retaining element
4a Engagement element of the retaining element, cam
4b Arm of the engagement element
4c Element
4d Connecting piece
5 Actuation element
5a First cam of the actuation element
5b Second cam of the actuation element
5c Recess of the actuation element
5d Extension
5e Extension edge
6 Holding device
6a External thread of the holding device
6b Longitudinal rib of the holding device
6c Injection needle unit connecting element
6d Latching arm
6e Stop
6f Recess
7 Multi-chamber cartridge, two-chamber cartridge
7a First plug
7b Second plug
7c Membrane
7d First chamber
7e Second chamber
7f Bypass
8 Blocking element
8a Stop
8b Recess
8c Blocking element edge
8d Blocking element support

What is claimed is:

1. A device for administering a dose of a fluid product, comprising:
a housing with a distal end and a proximal end or section, wherein a discharge stop of a dose to be administered is provided on the housing;
a pretensioned spring that is held at least partially by a drive element, wherein the drive element is supported axially in a distal direction such that the drive element can move in the housing to displace a plug held in a multi-chamber cartridge in the distal direction for administering the dose of the fluid product;
a retaining element for retaining the drive element loaded with a force of the spring, wherein, in a starting state of the device, the drive element can be held in a proximal position by an engagement between an engagement element provided on the retaining element and an engagement element provided on the drive element, wherein in a released state of the device, the engagement element of the retaining element is out of the engagement with the engagement element of the drive element; and
a discharge stop that is provided on the drive element, wherein the discharge stop of the drive element can be brought into a stop contact with the discharge stop of the housing for administering the dose of the fluid product, and the discharge stop of the drive element is arranged on the drive element offset axially in a proximal direction relative to the engagement element of the drive element,
wherein, in the starting state of the device, the retaining element is set apart in the proximal direction from the proximal end or section of the housing.

2. The device according to claim 1, wherein the discharge stop of the drive element is arranged on the drive element offset in a circumferential direction by 90 degrees relative to the engagement element of the drive element.

3. The device according to claim 1, wherein the drive element comprises a sleeve-shaped construction.

4. The device according to claim 1, wherein, in the starting state of the device, the proximal end of the drive element is in the stop contact with the housing.

5. The device according to claim 1, wherein the retaining element comprises a connecting piece that is arranged, at least in the starting state of the device, in a notch provided on the proximal end of the drive element.

6. The device according to claim 1, wherein an outer diameter of the drive element on a distal end of the drive element is different from an outer diameter on the proximal end of the drive element, and wherein the inner diameter of the drive element is the same on the distal end and on the proximal end of the drive element.

7. The device according to claim 6, wherein
the outer diameter of the drive element is greater on the proximal end than on the distal end of the drive element, and wherein the outer diameter of the distal end of the drive element is constructed such that the distal end of the drive element extends into the multi-chamber cartridge when the discharge stop of the drive element is in the stop contact with the discharge stop of the housing.

8. The device according to claim 1, wherein the engagement element of the retaining element is constructed as a cam and the engagement element of the drive element is constructed as a recess.

9. The device according to claim 1, further comprising an actuation element for activating the device, which can be moved relative to the housing.

10. The device according to claim 9, wherein the actuation element comprises a recess into which the engagement element of the retaining element can be extended in the released state of the device.

11. The device according to claim 1, further comprising a holding device for holding the multi-chamber cartridge, wherein the holding device is supported such that the holding device can rotate relative to the housing, wherein by rotating the holding device, contents of the multi-chamber cartridge can be mixed together.

12. The device according to claim 11, wherein the drive element comprises at least two retaining arms for retaining the multi-chamber cartridge in a defined position relative to the holding device.

13. The device according to claim 11, further comprising a blocking element for blocking the actuation element, wherein by rotating the holding device relative to the housing, the blocking element can be moved from a blocking position into a release position.

14. The device according to claim 13, wherein the holding device comprises a stop and the blocking element comprises a stop that interact for a movement of the blocking element from the blocking position into the release position.

15. The device according to claim 13, wherein the actuation element can be moved into the release position of the blocking element along a housing longitudinal axis relative to the housing, wherein the actuation element comprises an extension that can be moved in the release position of the blocking element axially in the distal direction into a recess of the blocking element.
